(19) United States
(12) Patent Application Publication
(10) Pub. No.: US 2026/0065479 A1
(43) Pub. Date: Mar. 5, 2026

(54) WELLNESS MANAGEMENT APPLICATION WITH AI-POWERED INFECTION DETECTION

(71) Applicant: LIGHT AI INC., Vancouver (CA)

(72) Inventors: Peter Douglas Whitehead, West Vancouver (CA); Udit Gupta, Surrey (CA); Lucas Krayacich, Calgary (CA)

(21) Appl. No.: 19/313,634

(22) Filed: Aug. 28, 2025

Related U.S. Application Data
(60) Provisional application No. 63/688,634, filed on Aug. 29, 2024.
(60) Provisional application No. 63/727,468, filed on Dec. 3, 2024.

(51) Int. Cl. G06T 7/00; G06T 7/11; G16H 40/67; G16H 80/00

(52) U.S. Cl. CPC G06T 7/0014; G06T 7/11; G16H 40/67; G16H 80/00; G06T 2200/24; G06T 2207/20081; G06T 2207/20084

(57) ABSTRACT

A wellness management application enables automated diagnosis of a throat infection based on a throat image. A captured image is segmented into a plurality of image segments corresponding to different anatomical structures. A set of machine learning models are applied to the respective image segments to generate respective prediction scores indicative of likelihood of infection. Each of the set of machine learning models are independently trained based on labeled images of the corresponding anatomical structure. The results of the respective models may be aggregated to generate an aggregate prediction. Furthermore, various visual representations may be generated that illustrate respective contributions of different regions of the image to the prediction. The wellness management application may be integrated with a telehealth system to facilitate diagnosis and treatment of infections.

### CROSS-REFERENCE TO RELATED APPLICATIONS

This application claims the benefit of U.S. Provisional Patent Application No. 63/688,634 filed on Aug. 29, 2024 and U.S. Provisional Patent Application No. 63/727,468 filed on Dec. 3, 3024, which are each incorporated by reference herein.

### BACKGROUND

The field of healthcare technology has seen significant advancements in recent years, particularly in the areas of remote diagnostics and telemedicine. With the widespread adoption of smartphones and other mobile devices, there is increasing potential for leveraging these technologies to improve access to healthcare services and enable more efficient diagnosis of common illnesses. The ability to capture and analyze medical imaging data using consumer devices could potentially revolutionize how individuals monitor their health and how healthcare providers deliver care. However, the development of accurate and reliable diagnostic tools that can be used by non-medical professionals presents numerous technical challenges, including issues related to image quality, data analysis, and the integration of artificial intelligence algorithms with consumer-grade hardware.

### BRIEF DESCRIPTION OF THE DRAWINGS

FIG. 1 is an example computing environment for operation of a wellness management application.

FIG. 2 is an example embodiment of a backend server that operates in conjunction with a wellness management application.

FIG. 3 is an example embodiment of a user client that operates in conjunction with a wellness management application.

FIG. 4 is an example operating scenario for use of a wellness management application.

FIG. 5 is an example inference module for inferring likelihood of infection from a throat image.

FIG. 6 is a flowchart illustrating an example embodiment of a process for generating

### DETAILED DESCRIPTION

A wellness management application enables automated diagnosis of a throat infection based on a throat image. A captured image is segmented into a plurality of image segments corresponding to different anatomical structures of the throat such one or more of the left tonsil, the right tonsil, the uvula, the tongue, the teeth, the soft palate, the hard palate, the gums, the inner linings of the lips, the inner lining of the cheeks, the oropharynx, full throat, or other anatomical structures. Alternatively, the image may be segmented into regions that do not necessarily depend on identifying anatomical structures (e.g., an image half, quadrant, central region, or other definable image region). A set of machine learning models are applied to the respective image segments to generate respective prediction scores indicative of likelihood of infection. Each of the set of machine learning models are independently trained based on labeled images of the corresponding anatomical structure. The results of the respective models may be aggregated to generate an aggregate prediction. Furthermore, various visual representations may be generated that illustrate respective contributions of different regions of the image to the prediction. The wellness management application may be integrated with a telehealth system to facilitate diagnosis and treatment of infections.

In some embodiments, the application is capable of detecting a variety of infections including, but not limited to, Group A Streptococcus (“GAS”), Nonspecific Viral Pharyngitis, Influenza, Respiratory Syncytial Virus, Mononucleosis, COVID-19, and Streptococcal Pneumonia. In some implementations, multiple instances of the inference pipeline may execute sequentially or in parallel (with respective independently trained machine learning models) to generate inferences for each of the different types of possible infections.

In certain embodiments, the wellness management application can be integrated into a digital health ecosystem, including those with applications in consumer wellness programs, healthcare provider services, and telemedicine. For consumer wellness, the application may empower individuals to manage health. This service can guide users on whether to seek medical attention or manage symptoms at home, potentially reducing unnecessary healthcare visits. For healthcare providers, the application can enhance decision-making, reduce diagnostic uncertainty, and optimize patient management. The application can be used to triage sore throats or other throat conditions in home or clinical settings, potentially reducing the need for unnecessary visits, improving patient interaction, optimizing visits, and improving diagnostic accuracy.

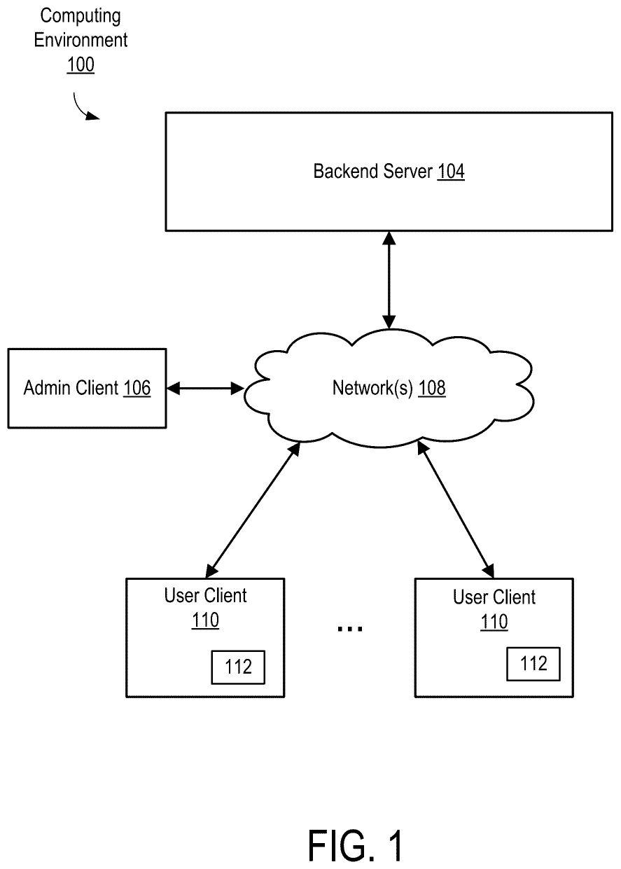

Referring to FIG. 1, an example embodiment of a computing environment 100 is illustrated. The computing environment 100 may include a backend server 104, an administrative client 106, one or more user clients 110, and a network 108. In some cases, the computing environment 100 may include different or additional components.

Embodiments of the described computing environment 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The user client 110 may comprise a computing device capable of capturing image data of a patient's throat using a built-in camera or an externally connected camera. The user client 110 may also be capable of communicating with the backend server 104 via the network 108. In some aspects, the user client 110 may connect with the backend server 104 via various communication protocols such as Bluetooth, WiFi, or any other wireless or wired communication protocol.

The user client 110 may execute a wellness management application 112 that may be locally installed on the user client 110 or may comprise a web-based application accessible via a web browser. The wellness management application 112 may include a user interface that enables various data entry for communicating to the backend server 104, transfer of image data from the user client 110 to the backend server 104, and viewing and/or interaction with various information obtained from the backend server 104 or directly inputted to the user interface. In various embodiments, the user client 110 may be embodied, for example, as a mobile phone, a tablet, a laptop computer, a desktop computer, or other computing device.

In an embodiment, the user interface of the wellness management application 112 may present various tracking data, analytics, and/or health-related recommendations based in part on the captured image data from the user client 110. For example, the wellness management application 112 may present various visualizations (e.g., graphs showing trends over time) of tracked health information. The wellness management application 112 may furthermore track general wellness activities, periods of rest, diet, or other aspects of patient health and wellness. The wellness management application 112 may furthermore present inferences relating to current health status and/or predictions of future health conditions based on the detected image data and other stored profile information for the patient. Predictions may relate to health conditions associated with specific anatomical targets in the throat. The predictions may furthermore recommend specific wellness measures such as what activities to adjust, rest, medication, etc. that are predicted to mitigate predicted conditions, prevent further illness, or avoid surgery. The user interface may furthermore recommend and monitor specific wellness regimens that reduce likelihood of illness.

In some embodiments, the user client 112 and wellness management application 112 may be utilized by a medical provider, e.g., in a clinical setting. Here, the wellness management application 112 may be coupled to interface with backend health systems of the medical facility such as electronic health records (EHR) databases. In other scenarios, the user client 112 may be used directly by an end user as a self-monitoring device. In some implementation, the user client 112 may enable telehealth services that are facilitated in association with wellness results obtained through the wellness management application 112.

The backend server 104 performs various functions for supporting training of machine learning models, performing inferences based on acquired biometric data or other health-related data, and generating user interface presentations in the user client 110. The backend server 104 may be implemented using cloud processing and storage technologies, on-site processing and storage systems, virtual machines, other technologies, or a combination thereof. For example, in a cloud-based implementation, the backend server 104 may include multiple distributed computing and storage devices managed by a cloud service provider. The various functions attributed to the backend server 104 are not necessarily unitarily operated and managed, and may comprise an aggregation of multiple servers responsible for different functions of the backend server 104 described herein. In this case, the multiple servers may be managed and/or operated by different entities. In various implementations, the backend server 104 may comprise one or more processors and one or more non-transitory computer-readable storage mediums that store instructions executable by the one or more processors for carrying out the functions attributed to the backend server 104 herein.

The administrative client 106 comprises a computing device for facilitating administrative functions associated with operation of the backend server 104. For example, the administrative client 106 may comprise a user interface for performing functions such as configuring parameters associated with various machine learning algorithms, initiating deployment of software updates to the user clients 110, etc. The user interface of the administrative client 106 may be embodied as an application installed on the administrative client 106 or may comprise a web-based application accessible via web browser.

The one or more networks 108 provides communication pathways between the backend server 104, the administrative client 106, and/or the user clients 110. The network(s) 108 may include one or more local area networks (LANs) and/or one or more wide area networks (WANs) including the Internet. Connections via the one or more networks 108 may involve one or more wireless communication technologies such as satellite, WiFi, Bluetooth, or cellular connections, and/or one or more wired communication technologies such as Ethernet, universal serial bus (USB), etc. The one or more networks 108 may furthermore be implemented using various network devices that facilitate such connections such as routers, switches, modems, firewalls, or other network architecture.

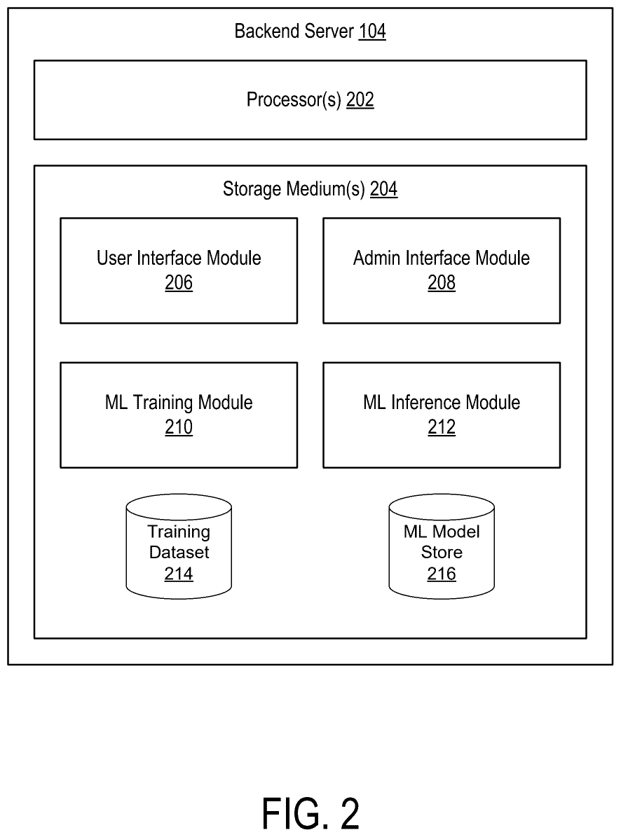

Referring to FIG. 2, an example embodiment of a backend server 104 is illustrated. The backend server 104 includes one or more processors 202 and one or more storage mediums 204. The one or more storage mediums 204 includes various functional modules (implemented as instructions executable by the one or more processors 202) including a user interface module 206, an administrative interface module 208, a training module 210, and an inference module 212. The storage medium 204 may furthermore store a training dataset 214 and a model store 216. In alternative embodiments, the backend server 104 may include different or additional modules. The one or more processors 202 and one or more storage mediums 204 are not necessarily co-located and may be distributed (e.g., in a cloud architecture).

The user interface module 206 facilitates server-side functions of a user interface of the wellness management application 112 accessible on the user clients 110. In some aspects, the user may input various information via a user interface on the user client 110 that is communicated to the user interface module 206. Furthermore, the user interface module 206 may output various analytical data, recommendations, or other information pertinent to monitoring human health.

In an embodiment, the user interface may enable input of various profile information for patients, information about the client device, and various other configuration settings. In some embodiments, input data from the user may be obtained interactively by presenting a series of questions via the user interface that enables structured input of data. Questions may be presented for various input forms such as multiple choice, true/false, or text-based inputs. Here, the user may enter various information about a patient such as physical characteristics (age, weight, etc.), health history, wellness regimen, diet, upcoming events, etc.

The user interface module 206 may furthermore facilitate presentation of various outputs from the backend server 104. For example, the user interface module 206 may output predictions about health and recommendations to mitigate effects of illness. The user interface module 206 may furthermore present various tracked data relating to user inputs about the patient's diet, wellness regimen, or other characteristics.

In some implementations, the user interface of the wellness management application 112 may execute locally on the user client 110. In this case, the user interface module 206 of the backend server 104 may include more limited functionality, such as facilitating updates, authorizing user credentials, accessing backend databases, etc. In other implementations, the wellness management application 112 may be implemented as a web application, in which case the user interface module 206 of the backend server 104 may directly generate interfaces via a web page for presentation in a browser of the user client 110.

The administrative interface module 208 facilitates various administrative functions associated with operation of the backend server 104. For example, the administrative interface module 208 may present an interface that enables configuration of various parameters of the machine learning module 210 (described below), controls versions and/or access to applications for the user clients 110, or performs other administrative functions.

The training dataset 214 stores various human health data associated with historical monitoring of human health. The training dataset 214 may include one or more cloud-based data sources and/or one or more locally accessible data sources. In some implementations, the training dataset 214 may comprise a centralized repository that may aggregate data from multiple different sources. In other implementations, the training dataset 214 may refer to two or more disparate data sources that may be managed by different entities and may be independently accessed by the backend server 104. The training dataset 214 may be accessible via an application programming interface (API) or may enable data to be downloaded via a web browser or other application.

The ML training module 210 trains one or more machine learning models based on a training dataset 214 that includes histories of monitored image data for different patients and their respective health histories. The training dataset 214 may furthermore include profile information for the various patients with tracked image data and health histories. The training dataset 214 may encompass data from patients with varying physical characteristics, wellness regimens, or other characteristics. Using various machine learning techniques, the ML training module 210 learns relationships between the monitored image data, profile information, and health outcomes to enable generation of health-related predictions and recommendations.

The model store 216 stores the one or more machine learning models generated by the training module 210.

The ML inference module 212 applies the one or more machine learning models to an input dataset to generate scores indicative of a likelihood of a health condition being present or occurring in the future. In an embodiment, the inputs to the ML inference module 212 may include stored profile data for a patient and a plurality of image data captured by the camera of the user client 110. The inferences may relate to specific health conditions, may relate to specific anatomical targets, and may relate to specific time frames for the condition to occur. For example, a prediction may indicate that a patient has an elevated risk of developing a throat infection in the next 10-14 days. The risk scores may comprise the likelihood values (e.g., expressed as a percentage or converted to a score on a predefined scoring scale), a classification of the likelihoods between different risk categories (e.g., low risk/high risk), or a combination thereof. The ML inference module 212 may furthermore generate recommendations for treating or mitigating a health condition. For example, the ML inference module 212 may predict that a period of rest (e.g., 3-5 days) may significantly reduce the likelihood of a throat infection.

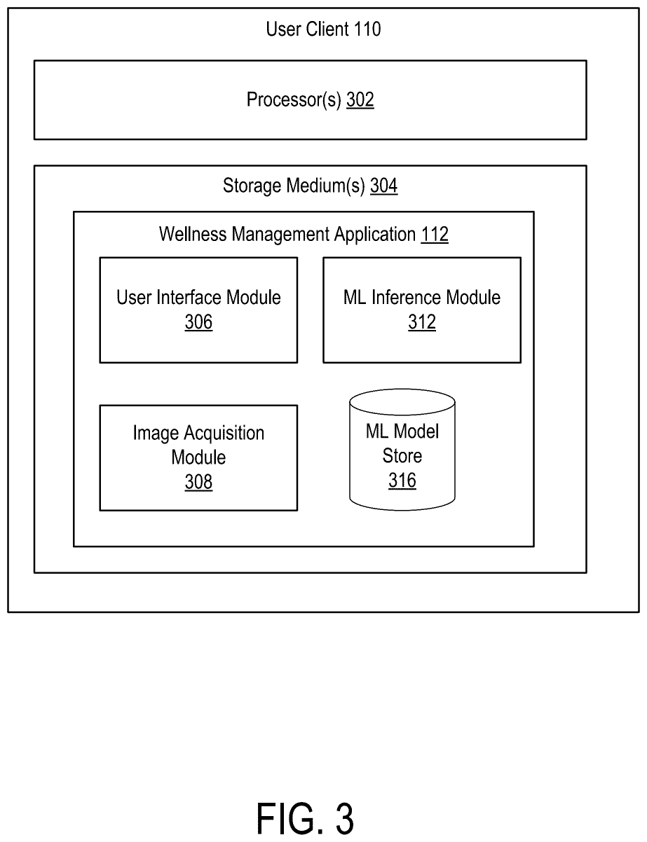

In operation, the ML inference module 212 may receive one or more raw images from the user client 110 and apply one or more machine learning models from the ML model store 216 to generate the inferences. Alternatively, the ML inference module 212 may receive a set of features from the user client 110 instead of the raw images (e.g., a feature vector). In alternative embodiments, the ML inference module 212 or a portion thereof may be implemented on the user client 110 instead of on the backend server 104, as shown in FIG. 3. In this case, the ML inference module 212 on the backend server 104 may be omitted.

Example embodiments of a system relevant to ML training and inference modules 210, 212 are described in U.S. Pat. No. 11,602,312 to Sarkaria et al., U.S. Pat. No. 11,369,318 to Sarkaria et al., and U.S. Patent Publication No. 2021/0295506 to Whitehead, et al., each of which are incorporated by reference in their entirety herein. The embodiments described herein may be integrated with any of the systems and/or devices described in the references above and may utilize any of the methods described therein to carry out the features described in this document.

FIG. 3 illustrates an example embodiment of a user client 110. The user client 110 includes one or more processors 302 and one or more storage mediums 304. The one or more storage mediums 304 includes various functional modules (implemented as instructions executable by the one or more processors 302) including a wellness management application 112 comprising a user interface module 306, an image acquisition module, and an inference module 312. The storage medium 304 may furthermore store a model store 316. In alternative embodiments, the user client 110 may include different or additional modules.

In this example, the ML inference module 312 locally executes on the user client 110 to perform inferences using or more ML models from the ML model store 316 that are suitable for edge deployment. Such models may be trained on the backend server 104 and may be reduced in size and complexity to enable deployment to the user clients 110. Alternatively, as described above, in other embodiments, inferences may be performed on the backend server 104. In this case, the ML inference module 312 may communicate with the ML inference module 212 of the backend server 104 (e.g., by sending raw images and/or feature vectors) to facilitate generation of inferences.

The image acquisition module 308 acquires images for analysis. For example, the image acquisition module 308 may interoperate with an integrated camera of the user client 110 or an externally attached camera. The image acquisition module 308 may alternatively enable access to an image store to retrieve previously captured images. In further embodiments, the image acquisition module 308 may enable extraction of individual frames or segments of video that can be analyzed using the techniques described herein.

The wellness management application 112 may also include a user interface module 306 for facilitating various client-side user interface functions as described herein.

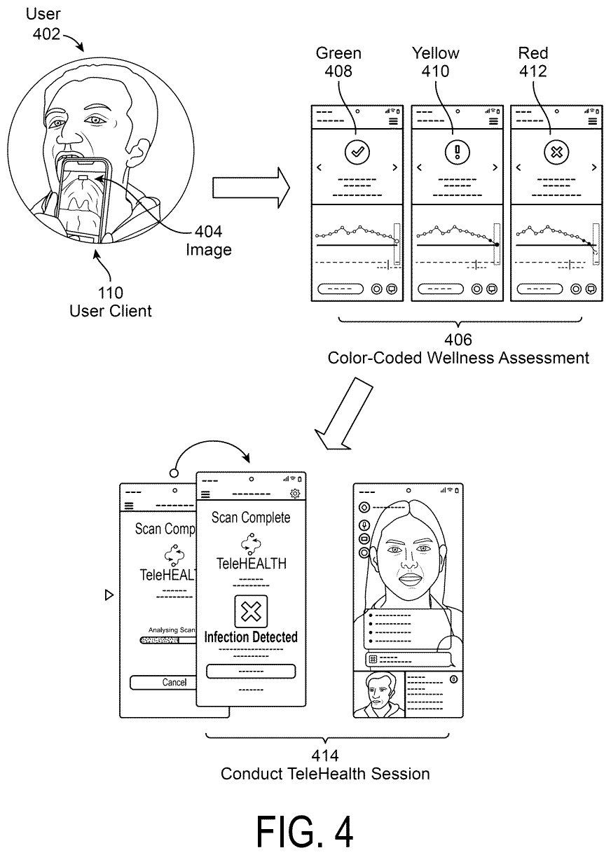

As shown in FIG. 4, the wellness management application 112 may provide a feature that allows a user 402 to capture an image 404 of their throat using a camera on a user client 110 (or an external camera), such as a smartphone or tablet. The application may provide instructions to the user on how to properly position the camera and capture the image 404. Once the image is captured, the application may process the image data, segmenting the image into objects for evaluation. For example, the image may be segmented into image segments corresponding to different potentially overlapping anatomical structures such as one or more of the left tonsil, the right tonsil, the uvula, the tongue, the teeth, the soft palate, the hard palate, the gums, the inner linings of the lips, the inner lining of the cheeks, the oropharynx, full throat, or other anatomical structures. Alternatively, the image may be segmented into regions independently of identified anatomical structure. The application 112 may then analyze the segmented image using a cloud-based convoluted neural network to detect patterns indicative of a throat-related illness. In some cases, the application 112 may provide a probability score indicating the likelihood of the presence of a throat-related illness based on the detected patterns in the image. This feature may enable users to perform preliminary health checks at home, potentially reducing the need for unnecessary healthcare visits.

In some embodiments, the wellness management application 112 may generate a weekly wellness assessment based on historic captured images, answers to questions, health history information, or other accumulated data. For example, the application 112 may facilitate capturing daily or weekly throat images and/or other health data to establish a baseline health state. The application 112 may present further questions for answering by the user based on the initial assessment. In some cases, the application may prompt the user to capture additional images of their throat using the camera of the user client 110. The application 112 may then analyze these images using the cloud-based convoluted neural network. If inputted information (images and/or other health data) deviates from the baseline, the application may issue an alert. For example, if the analysis indicates a high risk of infection, such as a likelihood score above a certain threshold, an output may be generated to indicate the predicted infection. In some embodiments, the application may provide a recommendation to the user to see a physician based on the predicted infection. This feature may enable users to monitor their health on a regular basis and take appropriate action when a potential health issue is detected.

In an example embodiment, the wellness management application 112 may provide a color-coded wellness assessment 406 that characterizes the risk level of a patient. The color-coding may be based on the probability score generated by the ML inferences. For example, a green color 408 may indicate a low risk of infection, a yellow color 410 may indicate a moderate risk, and a red color 412 may indicate a high risk. This color-coded wellness assessment 406 may provide a quick and intuitive way for users to understand their health status.

In some cases, the wellness management application 112 may also include graphs, charts, or other visual representations indicating how the health assessment changes over time. For instance, the application may display a line graph showing the probability score over a period of time, such as a week or a month. The line graph may be color-coded to match the wellness assessment, providing a visual representation of the user's health trend. This feature may enable users to monitor their health progress and identify any significant changes that may require medical attention.

In some embodiments, the wellness management application 112 may provide a feature that allows users to directly share their images and health assessments with a physician, insurance company, or other health provider. For example, the user may select an option in the application to send a report containing the captured images, the probability score, and other relevant health information to a specified recipient. The report may be sent via email, text message, or other communication methods supported by the user client 110. This feature may facilitate communication between users and health providers, potentially speeding up the diagnosis process and improving the efficiency of healthcare services.

In some aspects, the wellness management application 112 may also provide a feature that allows users to store their images and health assessments in a personal health record. The personal health record may be stored in the storage medium 204 of the backend server 104 or in a local storage of the user client 110. The personal health record may include a history of captured images, probability scores, wellness assessments, and other health information. This feature may enable users to keep track of their health history, which may be useful for future health assessments or medical consultations.

In some embodiments, the wellness management application 112 can be integrated with one or more telehealth services. This integration allows users to leverage the application's infection detection capabilities in conjunction with remote healthcare consultations. The application 112 may provide a feature that enables users to initiate a telehealth session 414 directly from within the app, particularly when the AI analysis indicates a potential infection or health concern.

For example, after performing the self-scan and obtaining results, the user may be prompted to initiate a telehealth session 414. When a user initiates a telehealth session 414, they may have the option to share their throat image and the AI-generated risk analysis with the telehealth provider. This sharing feature streamlines the consultation process by providing the healthcare professional with immediate access to relevant health data. The shared information may include the captured throat image, the AI-generated probability score indicating the likelihood of infection, and other pertinent patient health data stored in the user's personal health record.

In another implementation, a user may first initiate the telehealth session 414 before necessarily performing any scan. In the context of the telehealth session 414, the medical provider may initiate a scan request to prompt the user 402 to capture a throat image. The image may then be analyzed and the results sent to a device of the medical provider and/or to user client 110.

To ensure patient privacy and comply with healthcare regulations, the application 112 may implement a consent mechanism. Before any health information is shared with the telehealth provider, the user must explicitly grant permission. This consent process may involve a clear explanation of what information will be shared and how it will be used, followed by a user confirmation step.

The integration with telehealth services can significantly enhance the efficiency of remote consultations. By providing telehealth providers with AI-analyzed image data and health information prior to the consultation, the application enables more informed and focused discussions. This may lead to more accurate remote diagnoses and more effective treatment recommendations.

Furthermore, the combination of AI-powered infection detection and telehealth integration can facilitate the quick obtainment of prescriptions when necessary. If the telehealth provider determines that medication is required based on the shared data and virtual consultation, they may be able to issue electronic prescriptions directly through the integrated system. This streamlined process can reduce the time between symptom onset, diagnosis, and treatment initiation, potentially leading to faster recovery times for patients.

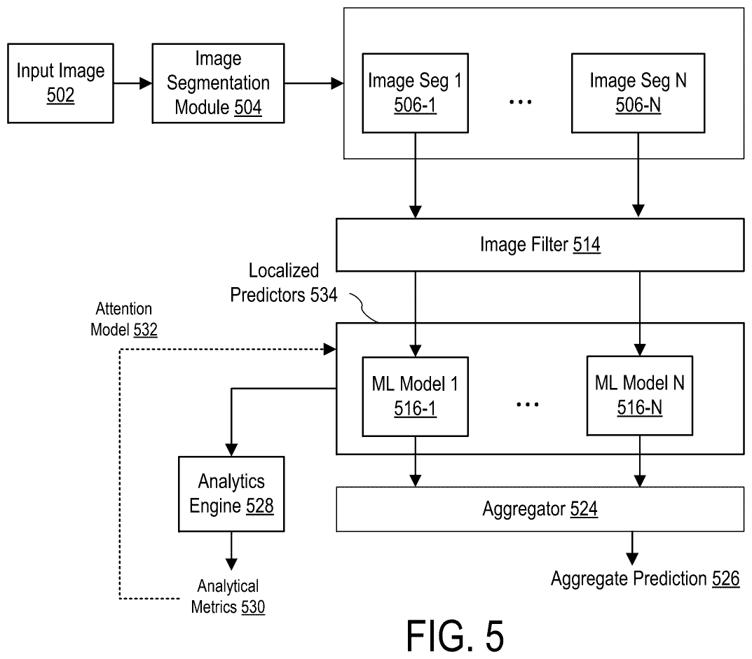

FIG. 5 illustrates an example inference module 500 for detecting infections based on throat images. The inference module 500 may execute on a backend server 104 (e.g., as ML inference module 212), on the user client 110 (e.g., as ML inference module 312), or a combination thereof.

In the illustrated approach, a captured input image 502 is first processed through a segmentation module 504 to segment the image into respective segmented images corresponding to different anatomical regions. For example, in one embodiment, the segmentation model operates to segment an image 502 of the throat into image segments 506 (e.g., image segments 516-1, . . . , 516-N). Each image segment 506 may be bounded around a different anatomical structure. The image segments 506 may overlap (i.e. include overlapping subsets of pixels). Furthermore, the anatomical structures corresponding to different image segments 506 may involve structures that may include all or part of one or more other structures. For example, a set of images segments 506 may correspond to the left tonsil, right tonsil, and uvula and another image segment 506 may correspond to the oropharynx which is inclusive of the tonsils and uvula. In various embodiments, the set of image segments 506 may correspond to one or more of the left tonsil, the right tonsil, the uvula, the tongue, the teeth, the soft palate, the hard palate, the gums, the inner linings of the lips, the inner lining of the cheeks, the oropharynx, the full throat (i.e., the full image) or other anatomical structures. In various embodiments, the image segments 506 may correspond to any sub-combination of these structures. In further embodiments, the image segments 506 may correspond to regions of the image that are not necessarily directly derived from a detected location of a specific anatomical structure and are instead derived from dividing the image according to some other segmenting function (which may include overlapping regions). For example the image segments 506 may correspond to one or more of a left half, right half, upper half, lower half, quadrant, central region, or other divided portion of the image.

Segmentation based on anatomical structures may be performed using any applicable image processing techniques. In one such implementation the image segmentation module 504 uses a machine learning-based approach in which a trained segmentation model is applied to the input image 502 to perform the segmentation. The segmentation model may be trained according to a supervised learning approach in which throat images in a training dataset are labeled to indicate the respective regions. For example, the segmentation model may comprise a trained neural network that operates to output pixel-wise classifications for each of the anatomical structures, and then produces bounding regions based on the pixelwise classifications. In another embodiment, a rule-based segmentation may be employed. In further embodiments, a combination of techniques may be used.

An image filter 514 determines whether each of the segmented images 540 meets a quality threshold. In one implementation, the image filter may 514 be implemented by applying a classifier that classifies each of the image segments 540 into one of a first class that meets the quality threshold and a second class that does not meet the quality threshold. This classifier may be trained using a training dataset of good images (i.e., images that meet the threshold) and bad images (images that do not meet the threshold) using supervised learning techniques. Alternatively, the classifier may be trained using unsupervised learning techniques trained on a set of good images only. In this case, the classifier may detect anomalous images that fail to meet similarity criteria relative to the good images. In other techniques, the image filter 514 may apply various processing rules that are not necessarily based on machine learning. For example, a rule-based image filter may determine quality based on various predefined characteristics such as resolution, contrast, blur, etc.

In one embodiment, multiple images 502 may be captured in a single session, individually segmented into the image segments 540, and processed through the image filter 514. In this case, the image filter 514 may be configured to select highest quality images for each image segment 506 from the set of images segments. In further embodiments, the user may capture a continuous video, and individual frames may be processed through the image segmentation module 504 and image filter 514.

A set of local predicators 534 may then be respectively applied to each of the segmented images 506 corresponding to each region (where respective quality thresholds are met via the image filter 514). The local predictors 534 may comprise respective machine learning models 516 (e.g., convolutional neural networks or other classifiers) that each output a prediction score indicative of the inferred likelihood of disease presence based on the respective image. For example, the prediction score may comprise a likelihood expressed as a value between 0 and 1, as a percentage, or a value on another predefined scale. Each of the machine learning models 516 may be trained using positive and negative training images specific to the segmented region. For example, a first model 516-1 may be trained on a set of training images of the left tonsil (which may be labeled to indicate whether an infection is present), a second model 516-2 may be trained on a set of training images of the right tonsil, etc. The respective models 516 may be independently trained such that they are each tailored for generating inferences for the corresponding type of image segment. For example, respective models 516 may be trained on images corresponding to one or more of the left tonsil, right tonsil, and uvula and another image segment 506 may correspond to the oropharynx which is inclusive of the tonsils and uvula. In various embodiments, the set of image segments 506 may correspond to one or more of the left tonsil, the right tonsil, the uvula, the tongue, the teeth, the soft palate, the hard palate, the gums, the inner linings of the lips, the inner lining of the cheeks, the oropharynx, the full throat (i.e., the full image) or other anatomical structures. Furthermore, models 516 may be trained on image segments corresponding to a left half, right half, upper half, lower half, quadrant, central region, or other divided portion of the image that is not necessarily derived directly from the detected location of an anatomical structure.

In one implementation, the local predictors 534 may comprise machine learning models 516 that may be smaller in size and utilize lower processing resources and memory allocation compared with a general model trained on the full image. Furthermore, the local predictors 534 may execute in parallel in some embodiments (e.g., using multiple processor cores) to enable efficient processing. In some embodiments, these models 516 may be suitable for edge deployment.

An aggregator 524 combines the predictions scores from the localized predictors 534 to generate an aggregate prediction 526. Different aggregation functions may be used. For example, the aggregation function may comprise a sum, average, weighted average, or other combining function that combines the respective scores from the localized predictors 534. The combined score may then be compared to one or more thresholds to generate the aggregate prediction 526. For example, the aggregate prediction 526 may comprise a binary output (i.e., yes or no for presence of infection). Alternatively, the aggregate prediction 526 may comprise a multi-level output (e.g., yes, no, or indeterminate for presence of infection). Alternative, the aggregate prediction 526 may comprise the combined score directly. In further embodiments, the combining function may first compare the individual scores from the localized predictors 534 against respective thresholds to generate binary outputs (e.g., represented as 0 or 1) or multi-level outputs (e.g., represented as predefined numeric values), and then combine these outputs to generate the aggregate prediction 526. For example, in one embodiment, the aggregate prediction 526 represents a consensus based on majority vote or other predefined threshold.

In an embodiment, the respective accuracies of each of the models used in the local predictors 534 may be characterized using a validation set. Model weights may then be applied in the aggregator 524 based on the respective accuracies, rates of false negatives, rates of false positives, or other parameters.

In an embodiment, the above-described approach may be specific to a type of infection (e.g., Strep A). In this case, multiple instances of the described inference module 500 may be employed to generate a set of predictions corresponding to the different types of infections. Each of these instances may include separately trained models tailored to the type of infection being detected. In other embodiments, a single model may be trained to output likelihoods associated with two or more different types of infections.

The analytics engine 528 generates one or more analytics metrics 530 associated with predictions. The analytics metrics 530 may provide supplemental information indicative of the confidence of the prediction and/or more fine-grained analysis indicative of which features of the image are predictive of infection or lack of infection, and/or relative strengths of those predictive contributions. In one implementation, the analytics engine 530 generates a heatmap indicative of the analytical metrics. Here, the method divides the image segment into a grid of regions (e.g., a set of pixels) within an image and generates sub-scores associated with respective regions indicative of the respective contributions of each portion of the region to either a positive or negative prediction. The sub-scores may be represented as a color-coded or grayscale coded image. These heatmaps may be outputted together with predictions as separate images in a user interface of the user client 110 or as an overlay on the original segmented image. Examples of suitable analytical techniques may include, for example, SHapley Additive explanations (SHAP) techniques, Gradient-weighted Class Activation Mapping (GRAD-CAM) techniques, a saliency map technique, attention layering technique, or other similar analytical techniques.

In one embodiment, the analytical metrics 530 may be applied to an attention model 532 that updates the localized predictors 534 by assigning varying importance (e.g., weights) to different features (e.g., regions of pixels) of the respective models. The attention scores may be based on sub-scores of the heatmaps described above.

The aggregated prediction 526 may be output as a signal to a user interface that causes the application 112 to display an output result indicative of the aggregate prediction. In some embodiments, the individual prediction scores and/or one or more heatmaps may also be output to the user interface. For example, the various outputs may be generated as a structured data object that includes, respective predictions scores, corresponding confidence intervals for each anatomical structure, statistical metrics, or other data that may be processed by the application 112 or other downstream applications (such as a telehealth application or service).

In an example heatmap, the image is divided into regions in which each region may be associated with a value that quantifies how much the region contributes to the prediction. For example, in one embodiment, a SHAP method is used in which the heatmap may comprise a color-coded representation that uses a first color (e.g., blue) to indicate regions that correspond to a negative prediction and a second color (e.g., red) to indicate regions that contribute to a positive prediction. The hue may furthermore be indicative of the value (e.g., with deeper hues corresponding to a higher contributions). In another embodiment, the heatmap may be generated using a Grad-CAM technique. In this example, the color-coded representation may be based on a color gradient that maps to different values indicative of the prediction contribution.

In further examples, the regions in the heatmap are not necessarily in the form of a grid. For example, the heatmap may spatially identify locations of image features (e.g., clusters of pixels) and assign values indicative of the contributions of those features to the predictions. In another example, the heatmap may be generated on a pixelwise basis that captures contributions of each pixel to the prediction. For example, in one embodiment, the heatmap comprises a saliency map.

In various implementations, the heatmap may be displayed side-by-side with the image being evaluated or may be overlaid on the image (e.g., using semitransparent color coding).

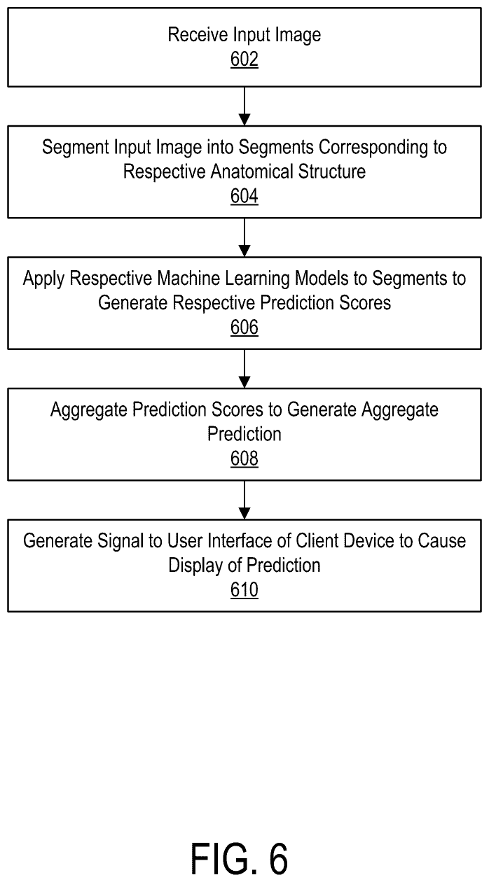

FIG. 6 is a flowchart illustrating an example embodiment of a process for automatically generating inferences representing likelihood of infection from a throat image. An input image is received 602 depicting a throat. The image may be captured from an image capture device that may be integrated with a user or from an external camera. The input image is segmented 604 into a plurality of image segments that each correspond to different respective anatomical structure of the throat (e.g., left tonsil, right tonsil, uvula, and oropharynx) (or alternatively, segments based on some predefined segmentation rule that does not necessarily depend on detecting anatomical structures). A respective machine learning model is applied 606 to each of the plurality of image segments to generate respective prediction scores each indicating likelihood of the presence of the infection. The respective machine learning models may be independently trained on training images associated with the respective anatomical structure or other type of image segment. The prediction scores are aggregated 608 to generate an aggregate prediction indicating an overall likelihood of infection. A signal is then generated 610 for a user interface of a user client device that causes the user client device to display an output result indicative of the aggregate prediction. In further embodiments, responsive to a positive result, a telehealth session may be facilitated in an automated manner. For example, a structured data object may be generated that includes the aggregate prediction score, individual predictions scores, and/or various statistical metrics derived therefrom, and the structured data object may be shared via a telehealth session to a device operated by a medical provider.

In contrast to conventional machine learning systems that apply a single model to an image, the disclosed approach improves computer functionality by segmenting the image into multiple segments based on identified characteristics (e.g., using an image segmentation model) and applying independent machine learning models to each segment. This segmentation and independent model application reduces computational overhead, improves training convergence, and enhances accuracy by tailoring models to features of individual image segments corresponding to different anatomical structures. Furthermore, the segmentation technique may enable each of the individual classifier models to be smaller size and less resource intensive than a single model, and may enable parallel processing if the respective image segments through the multiple models, thereby decreasing processing time and lowering memory allocation requirements. As a result, the system achieves improved scalability and efficiency, and yields more reliable predictions than approaches relying on a monolithic model.

Additionally, the described embodiments provide a technical improvement in facilitation of telehealth services by enabling efficient remote transmission, storage, and review of clinically relevant data. Rather than necessarily requiring an entire high-resolution image to be transmitted or manually interpreted by a remote clinician, the disclosed system automatically partitions the digital image into standardized segments and generates corresponding segment-based prediction scores indicative of likelihood of infection for those segments. This reduces bandwidth requirements during telehealth sessions while ensuring that downstream analysis may be performed by a telehealth provider. The disclosed embodiments thus improve computational efficiency and enhance the scalability and accuracy of telehealth workflows.

The disclosed embodiments furthermore constitute a technical improvement in medical treatment. By automatically segmenting digital medical images into standardized segments and applying independently trained machine learning models to each segment, the system generates prediction scores that directly supports therapeutic decision-making. For example, based on outputs provided by the inference module, a telehealth service may generate signals to a patient prescription database that enables dispensing of appropriate medication from a pharmacy.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention.

1. A method for automatically inferring presence of an infection based on a throat image comprising:
receiving an input image depicting a throat;
segmenting the input image into a plurality of image segments each corresponding to a different respective anatomical structure of the throat;
applying, to each of the plurality of image segments, respective machine learning models to generate respective prediction scores each indicating likelihood of the presence of the infection, wherein the respective machine learning models are independently trained on training images associated with the respective anatomical structure;
aggregating the prediction scores to generate an aggregate prediction indicating an overall likelihood of infection; and
generating a signal for a user interface of a user client device that causes the user client device to display an output result indicative of the aggregate prediction.

2. The method of claim 1, wherein the plurality of images segments corresponding to the different respective anatomical structures comprise image segments corresponding to one or more of: a left tonsil, a right tonsil, a uvula, a tongue, teeth, a soft palate, a hard palate, gums, an inner linings of lips, an inner lining of cheeks, an oropharynx, and a full throat.

3. The method of claim 1, further comprising:
generating one or more heatmaps indicative of respective contributions of different regions to the respective prediction scores; and
applying an attention function to update the respective machine learning models based on the one or more heatmaps.

4. The method of claim 3, wherein the one or more heatmaps comprises at least one of a SHapley Additive explanations (SHAP) heatmap, a Gradient-weighted Class Activation Mapping (GRAD-CAM) heatmap, an attention-based heatmap, and a saliency heatmap.

5. The method of claim 4, wherein generating the signal further comprises:
generating an output image depicting the input image with an overlaid color-coded representation of the one or more heatmaps.

6. The method of claim 1, further comprising:
responsive to the aggregate prediction indicating a positive detection of infection, generating a prompt in the user client device to initiate a telehealth call via a network-based telehealth service; and
responsive to receiving a selection of the prompt via the user client device, facilitating the telehealth call over a network connection.

7. The method of claim 6, wherein facilitating the telehealth call comprises transmitting, over the network connection to the telehealth service, at least one of: the input image, the aggregate prediction, and a heatmap indicative of contributions of different regions of the input image to the aggregate prediction.

8. A non-transitory computer-readable storage medium storing instructions for automatically inferring presence of an infection based on a throat image, the instructions when executed by one or more processors causing the one or more processors to perform steps including:
receiving an input image depicting a throat;
segmenting the input image into a plurality of image segments each corresponding to a different respective anatomical structure of the throat;
applying, to each of the plurality of image segments, respective machine learning models to generate respective prediction scores each indicating likelihood of the presence of the infection, wherein the respective machine learning models are independently trained on training images associated with the respective anatomical structure;
aggregating the prediction scores to generate an aggregate prediction indicating an overall likelihood of infection; and
generating a signal for a user interface of a user client device that causes the user client device to display an output result indicative of the aggregate prediction.

9. The non-transitory computer-readable storage medium of claim 8, wherein the plurality of images segments corresponding to the different respective anatomical structures comprise image segments corresponding to one or more of: a left tonsil, a right tonsil, a uvula, a tongue, teeth, a soft palate, a hard palate, gums, an inner linings of lips, an inner lining of cheeks, an oropharynx, and a full throat.

10. The non-transitory computer-readable storage medium of claim 8, further comprising:
generating one or more heatmaps indicative of respective contributions of different regions to the respective prediction scores; and
applying an attention function to update the respective machine learning models based on the one or more heatmaps.

11. The non-transitory computer-readable storage medium of claim 10, wherein the one or more heatmaps comprises at least one of a SHapley Additive explanations (SHAP) heatmap, a Gradient-weighted Class Activation Mapping (GRAD-CAM) heatmap, an attention-based heatmap, and a saliency heatmap.

12. The non-transitory computer-readable storage medium of claim 11, wherein generating the signal further comprises:
generating an output image depicting the input image with an overlaid color-coded representation of the one or more heatmaps.

13. The non-transitory computer-readable storage medium of claim 8, further comprising:
responsive to the aggregate prediction indicating a positive detection of infection, generating a prompt in the user client device to initiate a telehealth call via a network-based telehealth service; and
responsive to receiving a selection of the prompt via the user client device, facilitating the telehealth call over a network connection.

14. The non-transitory computer-readable storage medium of claim 13, wherein facilitating the telehealth call comprises transmitting, over the network connection to the telehealth service, at least one of: the input image, the aggregate prediction, and a heatmap indicative of contributions of different regions of the input image to the aggregate prediction.

15. A computer system comprising:
one or more processors; and
a non-transitory computer-readable storage medium storing instructions for automatically inferring presence of an infection based on a throat image, the instructions when executed by the one or more processors causing the one or more processors to perform steps including:
receiving an input image depicting a throat;
segmenting the input image into a plurality of image segments each corresponding to a different respective anatomical structure of the throat;
applying, to each of the plurality of image segments, respective machine learning models to generate respective prediction scores each indicating likelihood of the presence of the infection, wherein the respective machine learning models are independently trained on training images associated with the respective anatomical structure;
aggregating the prediction scores to generate an aggregate prediction indicating an overall likelihood of infection; and
generating a signal for a user interface of a user client device that causes the user client device to display an output result indicative of the aggregate prediction.

16. The computer system of claim 15, wherein the plurality of images segments corresponding to the different respective anatomical structures comprise image segments corresponding to one or more of: a left tonsil, a right tonsil, a uvula, a tongue, teeth, a soft palate, a hard palate, gums, an inner linings of lips, an inner lining of cheeks, an oropharynx, and a full throat.

17. The computer system of claim 15, further comprising:
generating one or more heatmaps indicative of respective contributions of different regions to the respective prediction scores; and
applying an attention function to update the respective machine learning models based on the one or more heatmaps.

18. The computer system of claim 17, wherein the one or more heatmaps comprises at least one of a SHapley Additive explanations (SHAP) heatmap, a Gradient-weighted Class Activation Mapping (GRAD-CAM) heatmap, an attention-based heatmap, and a saliency heatmap.

19. The computer system of claim 18, wherein generating the signal further comprises:
generating an output image depicting the input image with an overlaid color-coded representation of the one or more heatmaps.

20. The computer system of claim 15, further comprising:
responsive to the aggregate prediction indicating a positive detection of infection, generating a prompt in the user client device to initiate a telehealth call via a network-based telehealth service; and
responsive to receiving a selection of the prompt via the user client device, facilitating the telehealth call over a network connection.
